(19) United States
(12) Patent Application Publication
(10) Pub. No.: US 2004/0043462 A1
(43) Pub. Date: Mar. 4, 2004

(54) Chamber for in vivo screening of angiogenesis and tumor growth modulating compounds

(76) Inventors: Zishan Haroon, San Francisco, CA (US); Charles S. Greenberg, San Francisco, CA (US); Mark W. Dewhirst, Durham, NC (US)

Correspondence Address: JENKINS & WILSON, PA, 3100 TOWER BLVD, SUITE 1400, DURHAM, NC, 27707, US

(21) Appl. No.: 10/416,603

(22) PCT Filed: Nov. 13, 2001
(86) PCT No.: PCT/US01/47124

(51) Int. Cl. C12N 5/00; C12M 1/00

(52) U.S. Cl. 435/183; 435/283.1; 435/29; 435/375

(57) ABSTRACT

A chamber for in vivo delivery of an active agent, the chamber including a housing having at least two porous surfaces, the at least two porous surfaces disposed on substantially opposite sides of the housing from each other; an internal void space within the housing; and a matrix composition comprising an active agent, the matrix composition disposed within the internal void space. Therapeutic and screening methods employing the chamber are also disclosed, including methods for in vivo screening of angiogenesis and/or tumor growth modulating agents.

### CROSS REFERENCE TO RELATED APPLICATIONS

This application is based on and claims priority to U.S. Provisional Application Serial No. 60/248,156, filed Nov. 13, 2000, herein incorporated by reference in its entirety.

### TECHNICAL FIELD

The present invention pertains generally to methods and articles for in vivo screening of candidate compounds. More particularly, the present invention pertains to a chamber that can be used to study angiogenesis and/or tumor growth in vivo and to evaluate candidate compounds for an ability to modulate angiogenesis and/or tumor growth.

### Table of Abbreviations

AVM—arteriovenous malformation(s)

BPR—bovine pancreatic ribonuclease

BSA—bovine serum albumin

CT—computed tomography

DMEM—Dulbecco's Modified Eagle's Medium

DNA—deoxyribonucleic acid

FDA—United States Food and Drug Administration

F-ZC—fibrin Z-chambers

GEL—gelonin

gm—gram

GRO—growth regulated chemokine

h or hr—hour(s)

H & E—hematoxylin and eosin

HPF—high power focal

IL—interleukin

IL-1—interleukin-1

IL-6—interleukin-6

IL-8—interleukin-8

IL-12—interleukin-12

IP—intraperitoneal

IP-10—interferon-gamma induced protein 10 kD

IUPAC—International Union Of Pure And Applied Chemistry

kg—kilogram

kV—kilovolt(s)

MCP—monocyte chemoattracctant protein

mg—milligram

min—minute(s)

MIP—macrophage inflammatory protein

ml—milliliter

mM—millimolar

MT—Masson's trichrome

MVD—microvessel density

NAP-2 —neutrophil attractant/activation protein-2

NBF—neutral buffered formalin

ng—nanogram

nm—nanometer

nM—nanomolar

PBS—phosphate buffered saline

PET—positron emission tomography

PMSF—phenylmethylsulfonylfluoride

RES—reticular endothelial system

SOD—superoxide dismutase

TG—tissue transglutaminase

TGF—transforming growth factor

TGFβ—transforming growth factor-beta

TNFα—Tumor Necrosis Factor-alpha

TNF-β—Tumor Necrosis Factor-beta

T-ZC—tumor cell-containing chambers

μl—microliter(s)

VEGF—vascular endothelial growth factor

### BACKGROUND ART

The growth and proliferation of normal and abnormal tissue has been and continues to be an area of intense research activity. Angiogenesis plays a role in tissue growth, and in particularly wound healing as an example of tissue growth. As used herein, the term “angiogenesis” means the generation of new blood vessels into a tissue or organ.

Under normal physiological conditions, humans or animals undergo angiogenesis only in very specific restricted situations. For example, angiogenesis is normally observed in wound healing, fetal and embryonal development and formation of the corpus luteum, endometrium and placenta. Uncontrolled angiogenesis is associated with tumor metastasis (Folkman, J., N Engl J Med 28;333(26), 1757-1763 (1995)). Indeed, tumors have been loosely characterized in the art as wounds that do not heal. Dvorak, H. F., et al., Laboratory Investigation 57(6): pp. 673-686 (1987).

Both controlled and uncontrolled angiogenesis are thought to proceed in a similar manner. Endothelial cells and pericytes, surrounded by a basement membrane, form capillary blood vessels. Angiogenesis begins with the erosion of the basement membrane by enzymes released by endothelial cells and leukocytes. The endothelial cells, which line the lumen of blood vessels, then protrude through the basement membrane. Angiogenic stimulants induce the endothelial cells to migrate through the eroded basement membrane. The migrating cells form a “sprout” off the parent blood vessel, where the endothelial cells undergo mitosis and proliferate. The endothelial sprouts merge with each other to form capillary loops, creating the new blood vessel.

Persistent, unregulated angiogenesis occurs in a multiplicity of disease states, and abnormal growth by endothelial cells supports the pathological damage seen in these conditions. The diverse pathological disease states in which unregulated angiogenesis is present have been grouped together as angiogenic-dependent or angiogenic-associated diseases.

It is also recognized that angiogenesis plays a major role in the metastasis of a cancer. If this angiogenic activity could be repressed or eliminated, then the tumor, although present, would not grow. In the disease state, prevention of angiogenesis could avert the damage caused by the invasion of the new microvascular system. Therapies directed at control of the angiogenic processes could lead to the abrogation or mitigation of these diseases. However, the development of such therapies has been hindered by a lack of availability of assays that can be used to assess tissue growth, particularly tissue growth in an in vivo environment, and more particularly, angiogenesis in an in vivo environment.

To this end, there have been attempts to provide tissue growth assays, including angiogenesis assays, and articles for use in such assays. One such attempt is disclosed by Dvorak, H. F., et al., Laboratory Investigation 57(6): pp. 673-686 (1987). A chamber is employed in the assay method disclosed by Dvorak et al., and a representative embodiment of the chamber is shown in FIG. 1. Referring to FIG. 1, chamber 10 comprises a housing 12, a lower cover slip 14 and an upper cover slip 16. A plurality of regularly spaced pores 18, numbering only ten or less, are formed in upper cover slip 16 using a 20-gauge needle. Pores 18 are about 0.8 mm in diameter. A fibrin gel is added to the internal void space 20 of the chamber 10. Thus, pores 18 are present on only one side, which severely limits applications for chamber 10. Moreover, while angiogenesis was studied by Dvorak et al. via implantation of chamber 10 in an animal subject, the focus of the assay methods were for use in demonstrating the contributions of fibrinogen and related proteins to angiogenesis. Therefore, chamber 10 of Dvorak et al. has not been used and cannot be reliably used in a screen for an angiogenesis-modulating agent, because of limitations of size, limitations in the ability to analyze chamber contents, and design constraints. Additionally, chamber 10 of Dvorak et al. has never been used and indeed, cannot be reliably used to grow tumors or any other tissue because the presence of pores 18 on only one side creates long oxygen diffusion distances and limited access to blood vessels.

The current lack of an in vivo assay method has created a bottleneck in the drug development process at the pre-clinical and clincal stages. Thus, an in vivo tissue growth assay method represents a long-felt and continuing need in the art. Until the disclosure of the present invention presented herein, such screening assay method was not available in the art.

### SUMMARY OF THE INVENTION

A chamber for in vivo delivery of an active agent is disclosed. The chamber comprises: (a) a housing having at least two porous surfaces, the at least two porous surfaces disposed on substantially opposite sides of the housing from each other; (b) an internal void space within the housing; and (c) a matrix composition comprising an active agent, the matrix composition disposed within the internal void space. In a preferred embodiment, the matrix composition further comprises a tissue growth modulating agent. In a more preferred embodiment, the tissue growth modulating agent is fibrin, and the matrix composition further comprises a stabilizing agent.

An improved matrix composition that supports cell and/or tissue growth is also disclosed. The matrix composition comprises an effective amount of a tissue growth modulating agent and a stabilizing agent in an amount sufficient to retard degradation of the tissue growth modulating agent. In a preferred embodiment, the tissue growth modulating agent comprises fibrin. In a more preferred embodiment, the tissue growth modulating agent comprises fibrin and the stabilizing agent comprises phenylmethylsulfonylfluoride (PMSF), N-caproic acid, or combinations thereof.

A method of screening a candidate compound for tissue growth modulating activity is also disclosed. The method comprises: (a) providing a chamber comprising: (i) a housing having at least two porous surfaces, the at least two porous surfaces disposed on substantially opposite sides of the housing from each other, (ii) an internal void space within the housing, and (iii) a matrix composition comprising a tissue growth modulating agent, the matrix composition disposed within the internal void space; (b) implanting the chamber into a test animal; (c) administering a candidate compound to the test animal; (d) extracting the chamber after a time suitable for measurement of tissue growth; and (e) evaluating tissue growth in the chamber to thereby determine the tissue growth modulating activity of the candidate compound. In a preferred embodiment, the tissue growth modulating agent is fibrin, and the matrix composition further comprises a stabilizing agent.

A method of generating tissue growth in a vertebrate animal is also disclosed. The method comprises: (a) providing a chamber comprising: (i) a housing having at least two porous surfaces, the at least two porous surfaces disposed on substantially opposite sides of the housing from each other, (ii) an internal void space within the housing, and (iii) a matrix composition comprising an tissue growth modulating agent, the matrix composition disposed within the internal void space; (b) implanting the chamber in the vertebrate animal; and (c) generating tissue growth in the vertebrate animal through the implanting of the chamber.

Accordingly, it is an object of the present invention to provide a novel chamber for in vivo study of tissue growth, including but not limited to angiogenesis and tumor growth. The object is achieved in whole or in part by the present invention.

An object of the invention having been stated hereinabove, other objects will become evident as the description proceeds when taken in connection with the accompanying Figures and Laboratory Examples as best described herein below.

### BRIEF DESCRIPTION OF THE DRAWINGS

FIG. 1 is a top perspective view of a prior art chamber 10 as disclosed by Dvorak, H. F., et al., Laboratory Investigation 57(6): pp. 673-686 (1987).

FIG. 2 is a side elevation view of a chamber 110 of the present invention.

FIG. 3 is a cross sectional view along port 120 of chamber 110 of the present invention.

FIG. 4 is a cross sectional view along port 120 of chamber 110 of the present invention, wherein chamber 110 further comprises matrix composition 122.

FIG. 5 is a top perspective view of chamber 110 of the present invention.

FIG. 6 is a bottom perspective view of chamber 110 of the present invention.

FIG. 7 is a schematic diagram depicting the role of fibrin in wound healing.

FIG. 8 is a schematic diagram depicting the role of fibrin in wound healing and in tumor growth.

FIG. 9 is a line graph depicting that there was no significant body weight lost with SUGEN 5416 treatment and chamber implantation in test animals (solid line) as compared to control animals (broken line).

FIG. 10 is a bar graph depicting that SUGEN 5416 treatment causes significant tumor growth delay in test animals (shaded bar) as compared to control animals (open bar).

FIG. 11 is a bar graph depicting that microvessel density did not change with SUGEN 5416 treatment in tumor cell containing chambers of the present invention isolated from test animals (shaded bar) as compared to control animals (open bar).

FIG. 12 is a line graph depicting more residual D-dimer retention in tumor cell-containing chambers of the present invention isolated from test animals treated systemically with SUGEN 5416 (solid line) as compared to control animals (broken line).

FIG. 13 is a line graph depicting no significant body weight loss in animals treated systemically with SUGEN 5416 after implantation with a fibrin-containing chamber of the present invention (test animals=solid line; control animals=broken line).

FIG. 14 is a bar graph depicting that SUGEN 5416 inhibited granulation tissue formation in fibrin-containing chambers of the present invention implanted in test animals (shaded bar) as compared to fibrin-containing chambers of the present invention implanted in control animals (open bar).

FIG. 15 is a bar graph depicting that SUGEN 5416 inhibited neovascularization in fibrin-containing chambers of the present invention from test animals (shaded bar) as compared to fibrin-containing chambers of the present invention from control animals (open bar).

FIG. 16 is a line graph depicting an increase in residual D-dimer retention in fibrin-containing chambers of the present invention implanted in animals treated with SUGEN 5416 (solid line) as compared to fibrin-containing chambers of the present invention implanted in control animals (broken line).

FIGS. 17A and 17B are photographs showing gross examination of fibrin-containing chambers of the present invention as employed in Laboratory Examples 7-9, showing more influx of blood vessels in the controls (FIG. 17A) than SU5416 treated chambers that appeared paler in color (FIG. 17B). Fibrin is inherently pale yellow in color and lack of blood vessels in SU5416 treated chambers results in the paler appearance of the chambers. Arrows indicate apparent blood vessel growth.

FIGS. 17C and 17D are photomicrographs showing depth (represented by line with arrowheads at each end; bar in top right corner=100 microns) of granulation tissue developed inside fibrin-containing chambers of the present invention as employed in Laboratory Examples 7-9, which was used as a measure for the healing response. The granulation tissue in controls (FIG. 17C) is distinctly more than SU5416 treated chambers (FIG. 17D).

FIGS. 17E and 17F are photomicrographs showing that SU5416 treated fibrin-containing chambers of the present invention as employed in Laboratory Examples 7-9 had very thin stroma in the granulation tissue. MT stain for collagen (green) confirmed this observation as collagen was decreased in SU5416 treated chambers (FIG. 17F) than controls (FIG. 17E). Bar in top right corner=100 microns.

FIGS. 17G and 17H are photomicrographs showing that TG activity results in formation of isopeptide bonds that can be probed with a specific monoclonal antibody. Decreased isopeptide bond formation was found in extracellular matrix of SU5416 treated fibrin-containing chambers of the present invention as employed in Laboratory Examples 7-9 (FIG. 17H) in comparison to controls (FIG. 17G). Bar in top right corner=100 microns.

FIG. 18 is a graph of a TG activity (BP incorporation) assay showed more than 80% inhibition by SU5416 (x axis=concentration of SU5416 in micromolar, μM; y axis=% TG activity). There was more than 50% inhibition at levels effectively found in tissues treated with SU5416.

FIG. 19 depicts a Western blot for TG. Control tissues show the full length TG at 80 kd and multiple fragments that are typical in wound healing tissues for this enzyme. Tissues from SU5416 treated fibrin-containing chambers of the present invention as employed in Laboratory Examples 7-9 exhibit only one band for full length TG with no fragments suggestive of occupation of its nucleotide binding site.

FIGS. 20A and 20B are photographs showing gross examination of fibrin-containing chambers of the present invention as employed in Laboratory Example 10, showing more influx of blood vessels in the controls (FIG. 20A) than angiostatin treated chambers (1 μM angiostatin) that appeared paler in color (FIG. 20B). Fibrin is inherently pale yellow in color and lack of blood vessels in angiostatin treated chambers results in the paler appearance of the chambers.

FIGS. 20C and 20D are photomicrographs showing depth (represented by line with arrowheads at each end) of granulation tissue developed inside fibrin-containing chambers of the present invention as employed in Laboratory Example 10, which was used as a measure for the healing response. The granulation tissue in controls (FIG. 20C) is distinctly more than angiostatin treated chambers (1 μM angiostatin) (FIG. 20D).

FIG. 21 is a bar graph depicting that angiostatin inhibited granulation tissue formation in fibrin-containing chambers of the present invention implanted in test animals (shaded bar) as compared to fibrin-containing chambers of the present invention implanted in control animals (open bar). Scale=depth of granulation tissue (×10 microns).

FIGS. 22A and 22B are photomicrographs showing depth (represented by line with arrowheads at each end) of granulation tissue developed inside fibrin-containing chambers of the present invention as employed in Laboratory Example 11, which was used as a measure for effect on tumor growth. The granulation tissue in controls (FIG. 22A) is observably different than in SOD mimetic treated chambers (FIG. 22B).

FIG. 23 is a bar graph depicting the detection of a minor reduction in tumor growth after SOD mimetic administration in fibrin-containing chambers of the present invention implanted in test animals (horizontally hatched bar=SOD 201; cross hatched bar=SOD 150) as compared to fibrin-containing chambers of the present invention implanted in control animals (open bar). Scale=depth of granulation tissue (×10 microns); p<0.01.

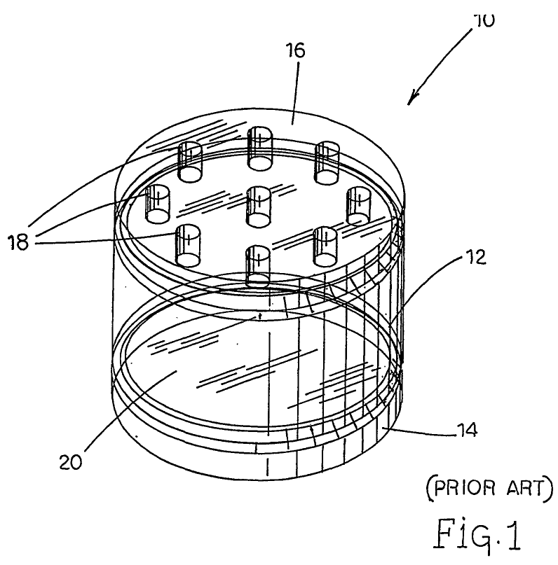

### DETAILED DESCRIPTION OF THE INVENTION

The present invention pertains to a chamber that can be used to deliver an active agent in vivo. In a preferred embodiment, the chamber is employed in an in vivo screening assay for novel compounds that modulate tissue growth, such as but not limited to compounds that enhance or inhibit angiogenesis and/or compounds that inhibit the growth of neoplastic tissue. A method of generating tissue growth in a vertebrate animal is thus also provided in accordance with the present invention.

The present invention also provides an in vivo screening assay method that can be used to identify compounds that enhance angiogenesis or to identify compounds that inhibit angiogenesis. Thus, significantly, the present invention provides the first in vivo assay that can be used to evaluate angiogenesis enhancing compounds that has been provided in the art. In a screen for angiogenesis modulating compound, a chamber of the present invention preferably comprises a fibrin containing matrix composition in that fibrin is a preferred tissue growth modulating agent when the desired tissue growth is blood vessel growth.

Additionally, the present invention provides an in vivo screening assay method for compounds that inhibit tumor growth or that inhibit the growth of new blood vessels to a tumor. Thus, the present invention provides a screening assay method that can also be used to identify antiangiogenic agents and antitumor growth agents.

In each embodiment of the assay method of the present invention, a candidate compound can be administered to a test animal subject either systemically or locally by including the candidate compound within the matrix composition in a chamber of the present invention. Thus, the chamber of the present invention provides for the use of small amounts of candidate compound, which can be very beneficial in the case of a rare, scarce and/or expensive candidate compound. Moreover, the ability to evaluate the activity of one or more candidate compounds in an in vivo setting can alleviate the currently observed bottleneck in the drug development process at the preclinical and clinical stages. Accordingly, the chamber and in vivo tissue growth assay method of the present invention solve a long felt and continuing need in the art.

A method of generating tissue growth in a vertebrate animal is also provided in accordance with the present invention. In a preferred embodiment, a chamber of the present invention comprises a fibrin containing matrix composition, and the matrix composition also comprises a cell or cells.

A. Definitions

The term “active agent” refers to compounds, molecules, or other substances that modulate, mediate, impart or otherwise affect responses or signals in a biological system in vitro or in vivo. A representative active agent comprises a tissue growth modulating agent. A cell can also comprise an active agent in that a cell is capable of sending and receiving chemotactic, chemokinetic and other biological signals and responses.

The term “tissue growth modulating agent” is meant to refer to an active agent that acts to stimulate or inhibit the growth of cells or tissues in culture or in vivo. Such an agent can thus also be referred to as a “tissue growth stimulating agent” or as a “tissue growth inhibiting agent”. A preferred “tissue growth modulating agent” is an active agent that modulates (i.e. stimulates or inhibits) angiogenesis. Such an agent is also referred to herein as an “angiogenesis modulating agent”, or depending on the activity of the agent, as an “angiogenesis stimulating agent” or as an “angiogenesis inhibiting agent”.

As used herein, the term “cytokine” refers to any secreted polypeptide that affects the functions of cells and is a molecule that modulates interactions between cells in the immune, inflammatory or hematopoietic response. A cytokine includes, but is not limited to, monokines and lymphokines, regardless of which cells produce them. For instance, a monokine is generally referred to as being produced and secreted by a mononuclear cell, such as a macrophage and/or monocyte. Many other cells however also produce monokines, such as natural killer cells, fibroblasts, basophils, neutrophils, endothelial cells, brain astrocytes, bone marrow stromal cells, epideral keratinocytes and B-lymphocytes. Lymphokines are generally referred to as being produced by lymphocyte cells. Examples of cytokines include, but are not limited to, Interleukin-1 (IL-1), Interleukin-6 (IL-6), Tumor Necrosis Factor-alpha (TNF-α) and Tumor Necrosis Factor beta (TNF-β).

As used herein, the term “chemokine” refers to any secreted polypeptide that affects the functions of cells and is a molecule which modulates interactions between cells in the immune, inflammatory or hematopoietic response, similar to the term “cytokine” above. A chemokine is primarily secreted through cell transmembranes and causes chemotaxis and activation of specific white blood cells and leukocytes, neutrophils, monocytes, macrophages, T-cells, B-cells, endothelial cells and smooth muscle cells. Examples of chemokines include, but are not limited to, interleukin-8 (IL-8), interleukin-12 (IL-12), neutrophil attractant/activation protein-2 (NAP-2), growth regulated chemokine (GRO) α, β and γ, interferon-gamma induced protein 10 kD (IP-10), macrophage inflammatory protein (MIP)-1a and-1b, and monocyte chemoattracctant protein (MCP) 1, 2 and 3.

The term “neoplasm” is meant to refer to an abnormal mass of tissue or cells. The growth of these tissues or cells exceeds and is uncoordinated with that of the normal tissues or cells and persists in the same excessive manner after cessation of the stimuli that evoked the change. These neoplastic tissues or cells show a lack of structural organization and coordination relative to normal tissues or cells that usually result in a mass of tissues or cells that can be either benign or malignant. Representative neoplasms thus include all forms of cancer, benign intracranial neoplasms, and aberrant blood vessels such as arteriovenous malformations (AVM), angiomas, macular degeneration, and other such vascular anomalies. As would be apparent to one of ordinary skill in the art, the term “tumor” typically refers to a larger neoplastic mass.

As used herein, neoplasm includes any neoplasm, including particularly all forms of cancer. This includes, but is not limited to, melanoma, adenocarcinoma, malignant glioma, prostatic carcinoma, kidney carcinoma, bladder carcinoma, pancreatic carcinoma, thyroid carcinoma, lung carcinoma, colon carcinoma, rectal carcinoma, brain carcinoma, liver carcinoma, breast carcinoma, ovary carcinoma, and the like. This also includes, but is not limited to, solid tumors, solid tumor metastases, angiofibromas, retrolental fibroplasia, hemangiomas, Karposi's sarcoma and the like cancers which require neovascularization to support tumor growth.

The phrase “treating a neoplasm” includes, but is not limited to, halting the growth of the neoplasm, killing the neoplasm, reducing the size of the neoplasm, or obliterating a neoplasm comprising a vascular anomaly. Halting the growth of the neoplasm refers to halting any increase in the size of the neoplasm or the neoplastic cells, or halting the division of the neoplasm or the neoplastic cells. Reducing the size of the neoplasm relates to reducing the size of the neoplasm or the neoplastic cells.

The terms “pharmaceutically acceptable”, “physiologically tolerable”, and grammatical variations thereof, as they refer to compositions, carriers, diluents and reagents, are used interchangeably and represent that the materials are capable of administration to or upon a vertebrate animal without the production of undesirable physiological effects such as nausea, dizziness, gastric upset and the like.

The term “candidate compound” or “candidate substrate” is meant to refer to any compound wherein the characterization of the compound's ability to modulate tissue growth, and preferably to modulate angiogenesis, is desirable. Exemplary candidate compounds or substrates include xenobiotics such as drugs and other therapeutic agents, carcinogens and environmental pollutants, as well as endobiotics such as steroids, fatty acids and prostaglandins.

The term “endothelium” means a thin layer of flat epithelial cells that lines serous cavities, lymph vessels, and blood vessels.

As used herein, the terms “target cell” and “target tissue” refer to a cell or to a tissue for which it is desired to produce a chemotactic, chemokinetic and other biological signal or response. For example, a “target tissue” can be a neoplastic tissue in which it is desired to retard or inhibit angiogenesis. Additionally, a “target tissue” can comprise a tissue in which stimulation of tissue growth is desired, such as an injured or diseased tissue. A “target cell” is thus preferably a cell within such tissues.

Following long-standing patent law convention, the terms “a” and “an” mean “one or more” when used in this application, including the claims.

B. Chamber

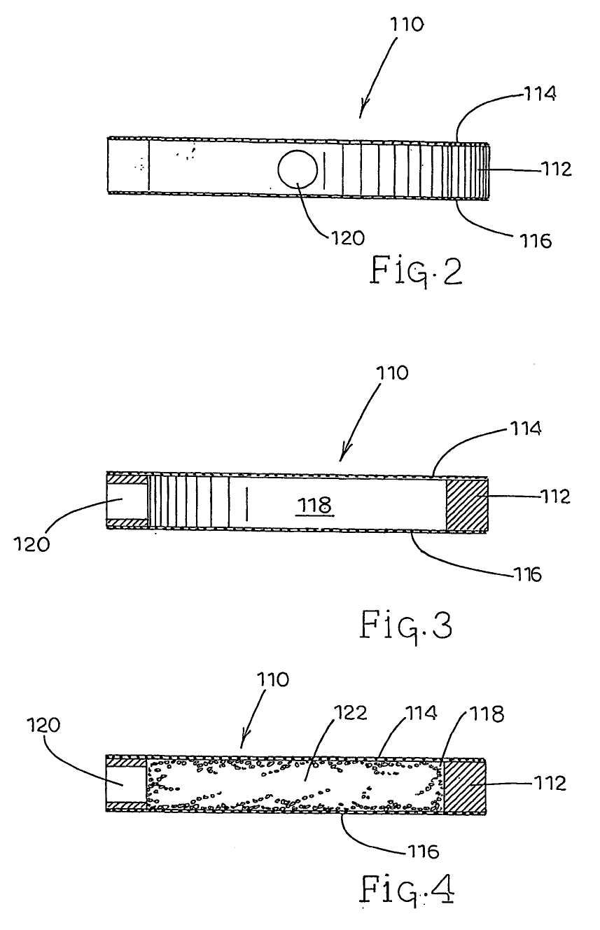

Referring now to the drawings, where like reference numerals refer to like parts throughout, a chamber of the present invention is referred to generally as 110. Referring now to FIGS. 2-6, chamber 110 comprises a housing 112. Chamber 110 further comprises at least two porous surfaces 114 and 116. Preferably, surfaces 114 and 116 are disposed on substantially opposite sides of housing 112. Housing 112 can thus serve as a support that is disposed between porous surfaces 114 and 116. Preferably, housing 112 comprises a ring. Thus, as shown in FIGS. 2-6, housing or ring 112 contacts porous surfaces 114 and 116 along a periphery of porous surfaces 114 and 116. Ring 112 can have an inside diameter ranging from about 5 to about 15 millimeters, and corresponding outside diameters ranging from about 6 to about 20 millimeters. Thus, ring 112 can have a width ranging from about 1 to about 5 mm. Preferably, ring 112 comprises an inert, non-immunogenic, non-pyrogenic material suitable for implantation into an animal subject. For example, ring 112 can comprise a metal (e.g., gold), a plastic material, a fiberglass material, a resinous material such as that sold under the registered trademark PLEXIGLAS® by Rohm & Haas of Philadelphia, Pa., or other suitable material.

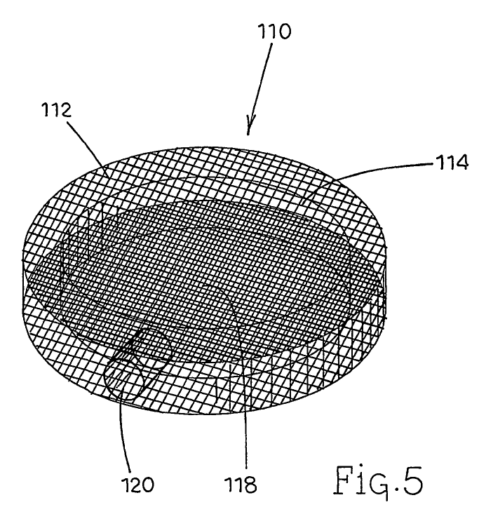

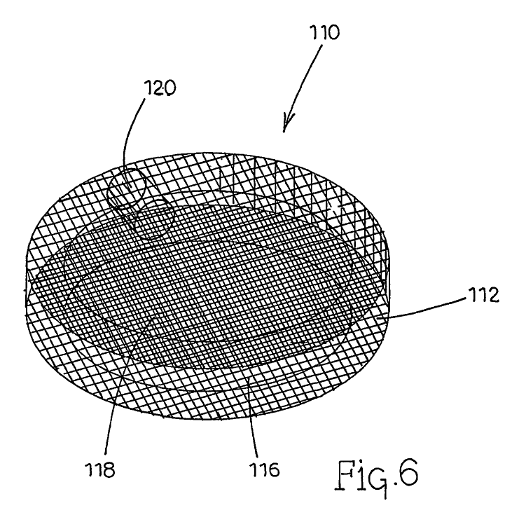

As best seen in FIGS. 5 and 6, surfaces 114 and 116 are preferably substantially permeable for the entire area of surfaces 114 and 116, i.e., an area extending substantially to the perimeter of each surface. Optionally, surfaces 114 and 116 can comprise a mesh. The mesh can comprise any inert, non-immunogenic, non-pyrogenic material suitable for implantation into an animal subject, such as nylon, cotton, polyester (e.g., that sold under the registered trademark DACRON® by E.l. du Pont de Nemours and Company of Wilmington, Del.), or other natural or other synthetic fiber. Preferred meshes comprise a pore size ranging from about 150 to about 200 micrometers. The use of a mesh facilitates removal of tissue from chamber 110, as discussed in the Laboratory Examples.

In FIGS. 2-6, chamber 110 further comprises an internal void space 118 defined by ring 112 and porous surfaces 114 and 116. Preferably, the depth of internal void space 118 ranges from about 1 to about 3 millimeters (which corresponds to the height of ring 112). Preferably, the depth of internal void space 118 is about 2 mm. Internal void space 118 is accessible via a port 120 that proceeds from the exterior of chamber 110 to internal void space 118 of chamber 110. The diameter of port 120 preferably ranges from about 1 millimeter to about 3 millimeters.

As best seen in FIG. 4, a matrix composition 122 is loaded into chamber 110 via port 120. Matrix composition 122 comprises an active agent as defined herein. Optionally, port 120 can be sealed closed, such as via an adhesive or heat, after loading matrix composition 122 into chamber 110. Alternatively, matrix composition 122 can be loaded into chamber 110 prior to attaching one of the porous surfaces 114 or 116 and port 120 can be omitted from chamber 110. Once the chamber 110 is prepared and loaded with matrix composition 122, it can be treated in accordance with any standard sterilization technique prior to use in an animal as described below.

B.1. Matrix Composition

As discussed above with respect to FIG. 4, chamber 110 comprises a matrix composition 122 that can optionally be loaded into chamber 110 via port 120. Matrix composition 122 comprises an active agent as defined herein. As disclosed in the Laboratory Examples, it is preferred that the matrix composition comprises a gel.

In an addition to an active agent, matrix composition 122 can comprise any suitable substrate or scaffolding, whether natural, synthetic or combination thereof, as would be apparent to one of ordinary skill in the art after review of the disclosure of the present invention. For example, biological substrates, including but not limited to collagen, laminin, agar, agarose, and the basement membrane derived biological cell culture substrate sold under the registered trademark MATRIGEL® by Collaborative Biomedical Products, Inc. of Bedford, Mass. comprise suitable substrate or scaffolding material. Synthetic matrix materials, substrate materials or scaffolding materials, which are typically made from a variety of materials such as polymers, are also within the scope of the present invention.

Matrix composition 122 can comprise any suitable growth media, buffer solutions, biological reagents or gelling reagents. A representative growth media is Dulbecco's Modified Eagle's Medium (DMEM), as disclosed in the Laboratory Examples. Thrombin is a representative biological reagent, as it is used to drive the formation of fibrin from fibrinogen. Indeed, component materials within a particular matrix composition 122 can be varied through the inclusion of different agents and combinations thereof as necessary to assess a particularly response or to accomplish a particular result.

Matrix composition 122 can further comprise a stabilizing agent so that the tissue growth modulating agent (e.g., fibrin) will not be degraded in an in vivo setting. For example, protease inhibiting agents can comprise the stabilizing agent and representative protease inhibiting agents include phenylmethylsulfonylfluoride (PMSF), N-caproic acid, aprotinin (a non-specific inhibitor) and plasminogen and activators 1 and 2 (specific protease inhibitors). Other suitable protease inhibitors will be apparent to one of ordinary skill in the art after review of the disclosure of the present invention presented herein.

The concentration of stabilizing agents is adjusted so that a tissue growth modulating agent, such as fibrin, remains stable in the matrix composition but the stability of the tissue growth modulating agent, such as fibrin, is not enhanced so as to prevent tissue growth processes from proceeding. For example, in a preferred embodiment, the concentration of the stabilizing agent for fibrin maintains the stability of fibrin while allowing remodeling of the fibrin to promote tissue growth in a chamber 110 of the present invention. As disclosed in the Examples PMSF and N-caproic acid comprise preferred stabilizing agents for a fibrin containing matrix composition 122 of the present invention. Typically, the PMSF is present in a molar concentration ranging from 0.5 to 50 μM PMSF, with 5 μM PMSF comprising a preferred molar concentration; and N-caproic acid is present in a concentration ranging from about 0.1 to about 10 mM, with a concentration of about 1 mM comprising a preferred concentration.

Preferably, the tissue growth modulating agent is present in an effective amount. As used herein, an “effective” amount refers to one that modulates (i.e. promotes or inhibits) tissue growth in a given setting, and preferably in an in vivo setting. After review of the disclosure herein of the present invention, one of ordinary skill in the art can tailor the effective amount according to desired parameters. By way of a particular example, an effective amount of fibrin preferably ranges from about 0.5 to about 10 mg/ml, more preferably from about 1 to about 7.5 mg/mL, and even more preferably from about 2.5 to about 5 mg/ml.

B.2. Active Agents

In a preferred embodiment, matrix composition 122 comprises a tissue growth modulating agent as an active agent. A preferred tissue growth modulating agent is an active agent that modulates (i.e. stimulates or inhibits) angiogenesis. Such an agent is also referred to herein as an “angiogenesis modulating agent”, or depending on the activity of the agent, as an “angiogenesis stimulating agent” or as an “angiogenesis inhibiting agent”. Representative tissue growth modulating agents include but are not limited to fibrin, fibrinogen, transforming growth factor (TGF), vascular endothelial growth factor (VEGF), chemokines, cytokines and/or any other growth factor as would be apparent to one of ordinary skill in the art after reviewing the disclosure of the present invention presented herein.

In a more preferred embodiment of the present invention, a tissue growth modulating agent in matrix composition 122 comprises fibrin. Preferably, fibrin is prepared in matrix composition 122 using reaction of thrombin and fibrinogen in a suitable medium, as disclosed in the Laboratory Examples.

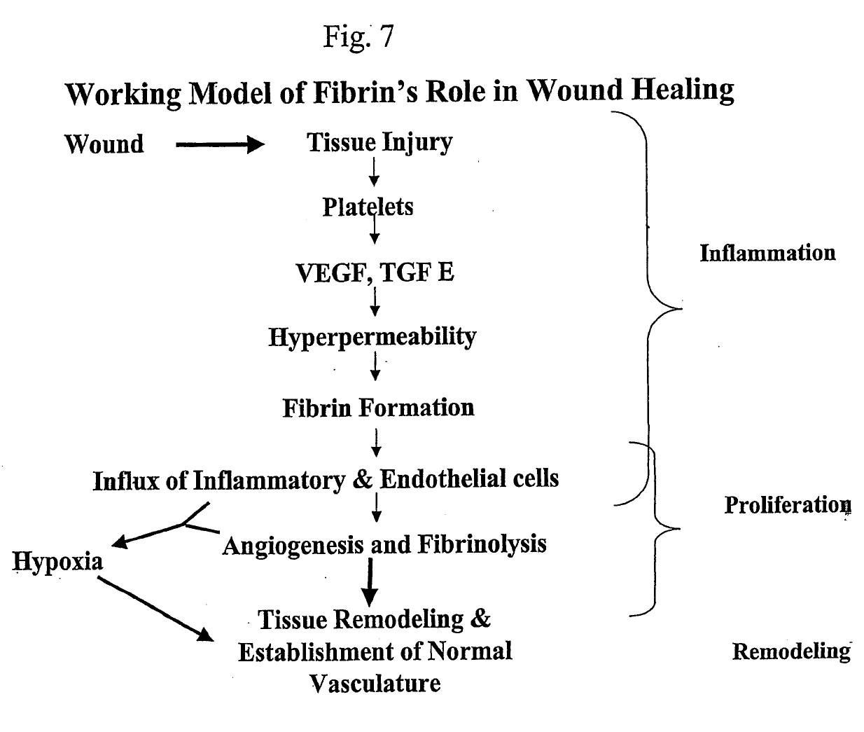

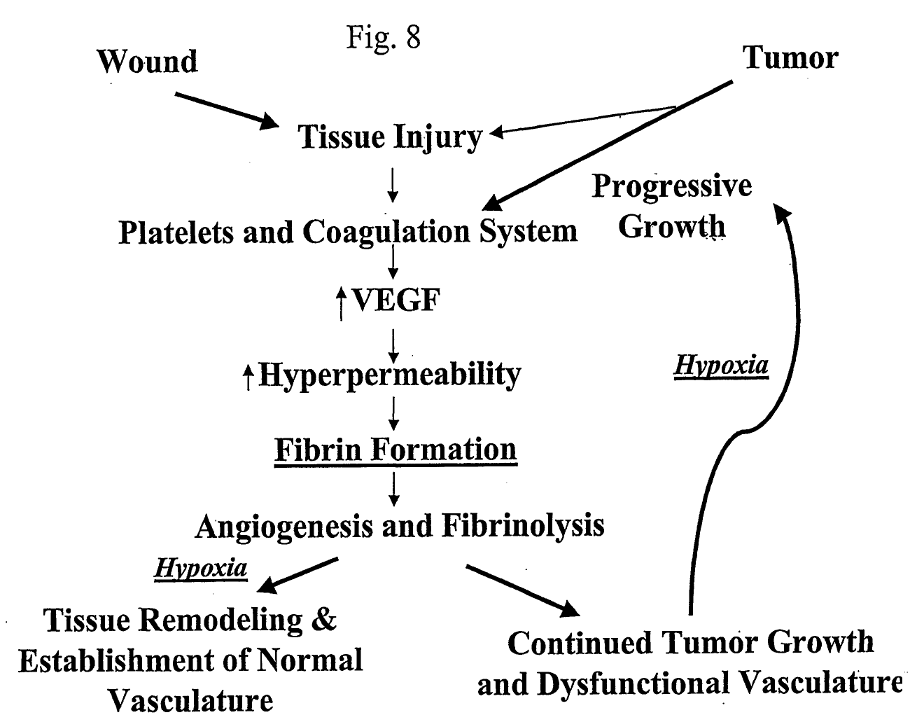

As depicted schematically in FIGS. 7 and 8, fibrin plays a wide-ranging role in wound healing and in tumor biology. First of all, it impedes blood loss. Additionally, fibrin induces platelets to release growth factors and trigger repair process. Fibrin also serves as a provisional matrix for cell migration and attracts other wound healing cells. Fibrin is also readily remodeled and promotes tissue retraction. In tumor biology, tumor cells induce fibrin formation by tissue factor pathway. Tissue factor is also induced by VEGF, while fibrin induces VEGF. VEGF alters vascular permeability and enhances fibrin formation.

Continuing with FIGS. 7 and 8, upon the occurrence of a wound and tissue injury, the platelet and coagulation system is activated. This induces increased levels and activity of VEGF and TGFβ, which then results in increased cellular hyperpermeability and fibrin formation from fibrinogen and thrombin. This leads to an influx of inflammatory and endothelial cells at the site of injury, followed by angiogenesis and fibrinolysis. Inflammation responses and cellular cell proliferation are also observed.

Continuing with FIGS. 7 and 8, the influx of inflammatory and endothelial cells and angiogenesis in fibrinolysis produce hypoxia (i.e., reduced level of oxygen as compared to basal levels), which drives tissue remodeling and establishment of normal vasculature. However, as best seen in FIG. 8, in the case of a tumor, continued tumor growth is observed along with the formation of dysfunctional vasculature. Thus, instead of tissue remodeling, the cycle repeats itself, and progressive growth of the tumor is observed. Thus, fibrin acts in autocrine and paracrine pathways that encourage tumor growth and angiogenesis.

A cell can comprise an active agent in that a cell is capable of sending and receiving chemotactic, chemokinetic and other biological signals and responses. Cells can be either naturally occurring cells or transfected cells produced in accordance art recognized techniques. Indeed, the preparation of recombinant vectors is well known to those of skill in the art and described in many references, such as, for example, Sambrook et al. (1992), Molecular Cloning: A Laboratory Manual (Cold Spring Harbor Laboratory Press, Cold Spring Harbor, N.Y.), incorporated herein in its entirety. The cell comprises a cell from a target tissue, such as a cell from a neoplasm or from a tissue in which the stimulation of growth is desired.

One will desire to prepare the active agent into a pharmaceutical composition, and thus the matrix composition can further comprise a suitable pharmaceutically acceptable carrier. Suitable pharmaceutical compositions in accordance with the invention will generally comprise an effective amount of the desired active agent admixed with an acceptable pharmaceutical diluent or excipient, such as a sterile aqueous solution, to give an appropriate final concentration with respect to the active agent. Such formulations will typically include buffers such as phosphate buffered saline (PBS), or additional additives such as pharmaceutical excipients, stabilizing agents such as bovine serum albumin (BSA), or salts such as sodium chloride.

The compositions are further rendered pharmaceutically acceptable by insuring their sterility, non-immunogenicity and non-pyrogenicity. Such techniques are generally well known in the art as exemplified by Remington's Pharmaceutical Sciences, 16th Ed. Mack Publishing Company (1980), incorporated herein by reference. It should be appreciated that endotoxin contamination should be kept minimally at a safe level, for example, less that 0.5 ng/mg protein. Moreover, for human administration, preparations should meet sterility, pyrogenicity, general safety and purity standards as required by United States Food and Drug Administration (FDA) Office of Biological Standards.

C. Method of Screening for Modulators of Tissue Growth

A method of screening a candidate compound for tissue growth modulating activity is also provided in accordance with the present invention. In a preferred embodiment, the method comprises: providing a chamber of the present invention; implanting the chamber into a test animal; administering a candidate compound to the test animal; extracting the chamber after a time suitable for measurement of tissue growth; and evaluating tissue growth in the chamber to thereby determine the tissue growth modulating activity of the candidate compound. In a more preferred embodiment, the tissue growth modulating activity comprises angiogenesis modulating activity or wound healing modulation activity. By the term “modulate”, and grammatical variations thereof, it is intended an increase, decrease, or other alteration.

Optionally, the tissue growth modulating agent is fibrin, fibrinogen, transforming growth factor, or combinations thereof. The matrix composition can further comprise a cell. The cell can comprise a cell from a target tissue, such as a neoplasm. Indeed, the test animal can further comprise a neoplasm, and in this case, the chamber can be implanted in the neoplasm.

The candidate compound can be systemically administered to the animal subject, or alternatively, the candidate compound can be administered in the chamber as a component of the matrix composition. The candidate compound can also be administered to the test animal by collecting serum from a human subject at a time after the human subject received a candidate tissue growth modulating compound, adding the serum to the chamber, and implanting the chamber in the animal subject.

In an alternative embodiment, the method can further comprise implanting two or more chambers into the test animal. The method can further comprise implanting two or more test chambers into the test animal, wherein a different candidate compound is inserted in each chamber. Thus, a rapid in vivo screen of multiple compounds is provided in accordance with the present invention.

As noted above, the chamber can be incubated in the test animal for any length of time, so long as the time is sufficient to provide for cell growth/wound healing processes to proceed. Preferably, the chamber is incubated in the test animal for about 5 to about 15 days, more preferably about 10 to about 12 days.

In a preferred embodiment, tissue is readily harvested from the chamber by cutting out the mesh surfaces of the chamber. Tissue invades and pervades the chamber due to the porous surfaces and thus, in situ conditions are closely approximated for histology and tumor biology analysis. The evaluation of tissue growth can be accomplished by any suitable or desired technique, including but not limited to: histology, immunohistochemistry, confocal imaging, magnetic resonance imaging, assessment of tumor growth, assessment of vascular density, immunoblotting, assessment of cell migration rate, assessment of cell death, assessment of hypoxia, assessment of vascular permeability, and combinations thereof.

A candidate substance identified according to the screening assay described herein has an ability to modulate tissue growth, and preferably has an ability to modulate angiogenesis. Such a candidate compound can have utility in the treatment of disorders and conditions associated with the biological activity abnormal tissue growth, including neoplastic growth. Candidate compounds can be hydrophobic, polycyclic, or both, molecules, and are typically about 500-1,000 daltons in molecular weight.

D. Method of Generating Tissue Growth

In accordance with the present invention, a variety of therapeutic methods are provided. In one embodiment, a method of delivering an active agent to a vertebrate animal is provided. The method comprises providing a chamber as disclosed herein; and delivering the active agent to the vertebrate animal by implanting the chamber in the vertebrate animal.

A method of generating tissue growth in a vertebrate animal is also disclosed. The method comprises: providing a chamber of the present invention; implanting the chamber in the vertebrate animal; and generating tissue growth in the vertebrate animal through the implanting of the chamber. In one embodiment, the tissue growth that is generated comprises angiogenesis.

Preferably, the active agent comprises a cell, a tissue growth modulating agent, or combinations thereof. The cell can comprise a cell from a target tissue. The tissue growth modulating agent can optionally comprises fibrin, fibrinogen, transforming growth factor, a chemokine, a cytokine or combinations thereof.

A preferred subject is a vertebrate animal subject. A preferred vertebrate animal is warm-blooded vertebrate animal, and a preferred warm-blooded vertebrate animal is a mammal. A preferred mammal is a human. Thus, as used herein, the term “patient” is includes both human and animal patients, and veterinary therapeutic uses are provided in accordance with the present invention.

Warm-blooded vertebrate animals comprise preferred subjects for treatment in accordance with the methods of the present invention. Therefore, the invention concerns mammals and birds.

Contemplated is the treatment of mammals such as humans, as well as those mammals of importance due to being endangered (such as Siberian tigers), of economical importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), and horses. Also contemplated is the treatment of birds, including the treatment of those kinds of birds that are endangered, kept in zoos, as well as fowl, and more particularly domesticated fowl, i.e., poultry, such as turkeys, chickens, ducks, geese, guinea fowl, and the like, as they are also of economical importance to humans. Thus, contemplated is the treatment of livestock, including, but not limited to, domesticated swine (pigs and hogs), ruminants, horses, poultry, and the like.

In accordance with the present invention, a method of generating new tissue growth in a vertebrate animal subject is provided. Cells from a tissue or organ can be incorporated into a chamber of the present invention along with a matrix composition comprising, for example, fibrin: Representative tissues include pancreas, liver, or other suitable tissues that will be apparent to one of ordinary skill in the art after review of the disclosure of the present invention herein. The chamber loaded with a matrix composition, the matrix composition comprising a cell and fibrin, can be implanted in any suitable location (e.g., subcutaneously or within a target tissue) within a vertebrate animal. The matrix composition facilitates the recruitment of cells to the chamber to generate tissue growth within the chamber.

Preferably, the cells that are included in the chamber are cells from the vertebrate animal subject so as to minimize problematic immunological responses to the chamber. Stated differently, the vertebrate animal subject recognizes the cells within the chamber as “self” as opposed to “non-self”, and thus, problematic immune responses are avoided. The internal void space of the chamber also facilitates new tissue growth by providing an enclosed space and scaffolding upon which tissue growth can occur.

As noted above, in a preferred embodiment of a chamber of the present invention, the matrix composition comprises fibrin. A matrix composition comprising fibrin can also be used in the treatment of cardiovascular disease. A chamber loaded with matrix composition comprising fibrin can be implanted at a site of cardiovascular disease. New blood vessel growth is generated at the site of implantation of the chamber to thereby treat the cardiovascular disease at the site of implanting of the chamber.

### LABORATORY EXAMPLES

The following Laboratory Examples have been included to illustrate preferred modes of the invention. Certain aspects of the following Laboratory Examples are described in terms of techniques and procedures found or contemplated by the present co-inventors to work well in the practice of the invention. These Laboratory Examples are exemplified through the use of standard laboratory practices of the co-inventors. In light of the present disclosure and the general level of skill in the art, those of skill will appreciate that the following Laboratory Examples are intended to be exemplary only and that numerous changes, modifications and alterations can be employed without departing from the spirit and scope of the invention.

### Laboratory Example 1

### Chamber Preparation

Customized PLEXIGLAS® rings are prepared. The rings have internal diameters of 10 and 5 mm and external diameters of 14 and 8 mm for rats and mice, respectively. A 1 mm port is formed on a lateral surface of the ring and the port is used to load the ring with matrix composition.

A small amount of MF™ cement (available commercially, such as from MF Composites, Inc., of Mississauga, Ontario, Canada) is applied on the ring surface. The ring is then pressed against the nylon mesh (available commercially, such as from Millipore Corporation of Bedford, Mass.) and allowed to dry for about 4 hours. The chamber is then cut from the nylon mesh around the periphery of the ring to produce a PLEXIGLAS® ring with one side covered with nylon mesh. The procedure is repeated for the other side. The nylon mesh is inspected for proper bond with the rings. The chambers are then sent for gas sterilization, or other suitable sterilization.

### Laboratory Example 2

### Matrix Composition Preparation

Materials:

Fibrinogen, Plasminogen depleted (#341578 (bottle of freeze dried powder), Calbiochem of La Jolla, Calif.)

Phenylmethylsulfonylfluoride (PMSF) (A8456, Sigma Chemical Company of St. Louis, Mo.)

N-Caproic acid (A2504, Sigma Chemical Company of St. Louis, Mo.)

Dulbecco's Modified Eagle's Medium(DMEM) (#11995-065, GibcoBRL of Rockville, Md.)

Thrombin 1 Unit/ul

Preparation:

53 milliliters (mls) of DMEM with 5 μM PMSF and 1 mM N-Caproic acid is prepared as a control solution. 25 mls of this solution is added to a whole bottle of fibrinogen freeze-dried powder and incubated at 37° C. for 1 hour. The solution is then transferred to a 50 ml conical tube and spun at 3000 rpm for 5 minutes for use as a test solution.

The concentration of fibrinogen is measured by spectrophotometer. 50 ul of test and control solutions are diluted in 5 ml of de-ionized water. The diluted test solution is measured at 325 nanometers (nm) and 280 nm with control solution as a blank. The reading at 280 nm is subtracted from that at 325 nm. The result is multiplied by 50.5 and 2, and then divided by 1.62 for final fibrinogen concentration. The fibrinogen is diluted to about 5 mg/ml for experiments.

### Laboratory Example 3

### Chamber Loading

The sterilized chambers are placed on sterilized 20 millimeter diameter caps with the port of the chambers facing to the right side. The caps provide a flat surface upon which the chambers are placed during loading to minimize leakage. A 1 ml tuberculin syringe with 20-gauge needle is used to fill the chambers. The chamber is held from the sides and pressed against the cap. The fibrinogen solution is added from the right side through the port. The solution is followed with 2-8 μl of thrombin from a pipette. The chamber, or fibrin Z-chamber (F-ZC) is allowed to stand for about 15 minutes so the fibrinogen can gel into fibrin. Preferably, the chambers are implanted soon after preparation.

A tumor cell chamber, or tumor Z-chamber (T-ZC) is prepared by adding tumor cells to the fibrinogen solution before adding the solution to the chamber. The tumor cells are embedded in fibrin gel.

### Laboratory Example 4

### Implantation of the Chambers

150-200 gm rats or 20-25 gm mice are preferably used for implantation. The animals are anesthetized with ketamine and ⅔^{rd} standard dosage of phenobarbital (preferably that sold under the registered trademark NEMBUTAL® by Abbot Laboratories of Abbot Park, Ill.). An additional 0.5 ml (rats) and 0.1 ml (mice) of saline intraperitoneally (IP) is also preferably administered. The animals' backs are shaved and they are surgically prepped with isopropyl alcohol and HIBICLENS® wipes, which are available from Zeneca, Limited of London, England, United Kingdom.

Implantation is accomplished by making two 2 cm long incisions on the backs of the animals, followed by making blunt dissections on both sides to establish a pocket in the subcutaneous space. The chambers are inserted into these pockets. Clips are used to close the incision in rats and sutures are used for mice. A topical antibiotic, such as that sold under the registered trademark NEOSPORIN® by Glaxo-Wellcome, Inc., is applied over the incision.

To harvest the chambers, a long incision is made on the backs of the animals. The surrounding fascia is observed and noted. The chambers are pushed from outside the animal and pulled out. Pictures are taken at this stage to observe differences, if any, between tissue within and encompassing the chamber and the surrounding tissue. The mesh and issue agglomerated thereon are cut from the chamber with scalpel (preferably a #11 blade) and then processed in accordance with a desired analysis approach.

For example, for paraffin treatment, the sample can be put into a paraffin cassette and fixed in 10% neutral buffered formalin (NBF) for 24-36 hours. To freeze a sample, a drop of OCT can be added on top and then the sample can be partially fixed in liquid nitrogen. Then the sample is cut in half, embedded face down in OCT in a cryomold, and frozen with liquid nitrogen. For a Western blot, the nylon mesh is pulled apart and the tissue is scraped from the mesh and saved in a propylene tube after freezing it in liquid nitrogen.

### Laboratory Example 5

### Use of Chambers to Assess Tumor Growth after Systemic Treatment with an Angiogenesis Inhibiting Agent

Chambers, or T-ZC, containing a matrix composition comprising fibrin and tumor cells were used to assess tumor growth in response to systemic treatment with the angiogenesis inhibiting agent SUGEN 5416. SUGEN 5416 is described by Mendel et al., (2000) Anti-Cancer Drug Design 15:29-41, and its chemical name is 3-(2,4-Dimethylpyrrol-5-yl)methylene-2-indolinone. SUGEN 5416 was administered at a concentration of 20 mg/kg.

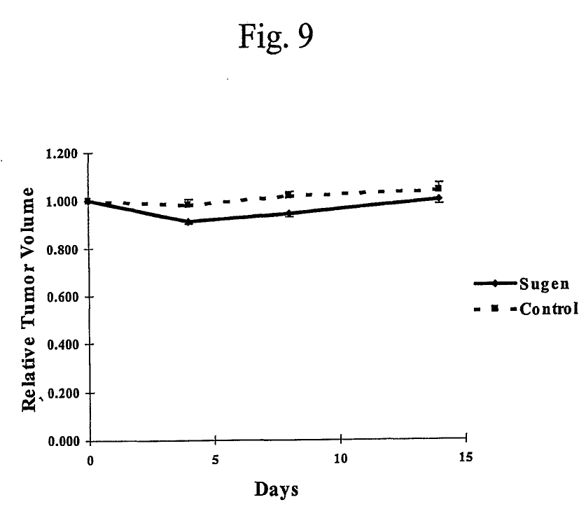

Referring now to FIG. 9, data are presented in a line graph format and depict that no significant body weight loss in the test animals occurred with SUGEN 5416 treatment and chamber implantation has compared to control (i.e., untreated) animals. The data are depicted as relative tumor volume as compared to days of implantation, and the test and control animals compare favorably.

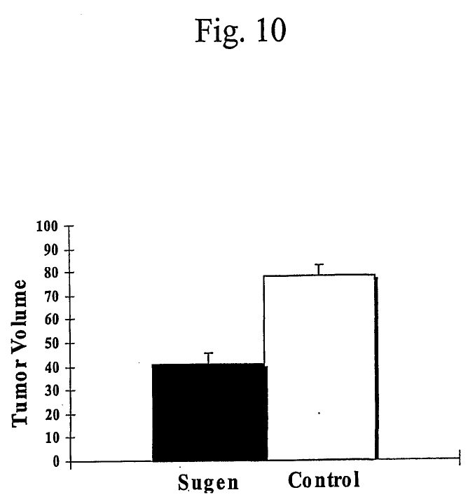

Referring now to FIG. 10, it was observed that SUGEN 5416 treatment caused significant tumor growth delay, with p values of less than 0.0001. Particularly, SUGEN 5416 treatment retarded tumor growth so that only a 40% increase in tumor volume was observed in test animals as compared to about a 90% increase in tumor volume in control animals.

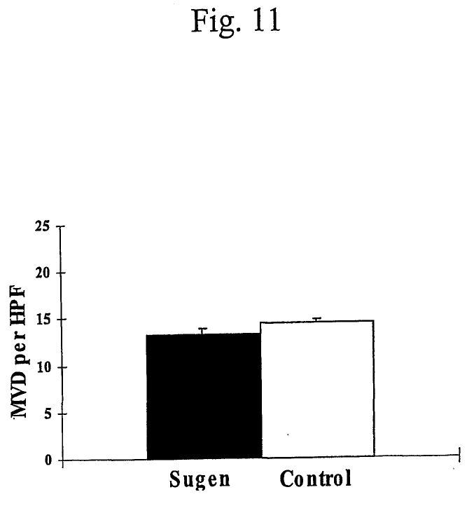

Referring now to FIG. 11, it was observed that microvessel density (MVD) did not change with SUGEN 5416 treatment in the tumor cell-containing chamber, with a statistical p value=0.11. As shown in FIG. 11, SUGEN 5416 treatment produced an observed MVD per high power (200×) focal (HPF) of about 13, while in the control animals, MVD per HPF of about 15 was observed. These data were also confirmed through visible inspection of tissue from each sample where the tissue was viewed under a microscope at 5× magnification.

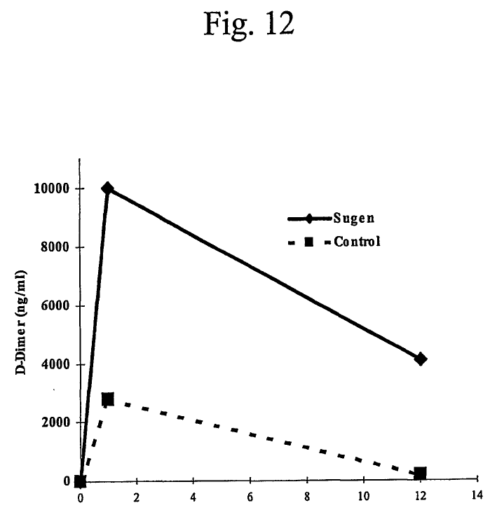

Referring now to FIG. 12, more residual D-dimer retention was observed in tumor cell-containing chambers T-ZC from the test animals treated with SUGEN 5416 than was observed in tumor cell-containing chambers T-ZC from control animals. To elaborate, it is recognized in the art that newly (aid fibrin is degraded to D-dimer by invading endothelial and tumor cells. D-dimer reenters circulation through the neovessels. As shown in FIG. 12, residual D-dimer retention amounts were greater in the chambers that were implanted and removed from animals that were treated with the SUGEN 5416 as compared to chambers that were implanted and removed from control animals. The data presented in FIGS. 9-12 thus establish an aspect of the utility of the chambers of the present invention in analyzing the effects of an angiogenesis inhibiting agent in an in vivo setting.

### Laboratory Example 6

### Use of Chambers to Assess Wound Healing in Response to Systemic Treatment with an Angiogenesis Inhibiting Agent

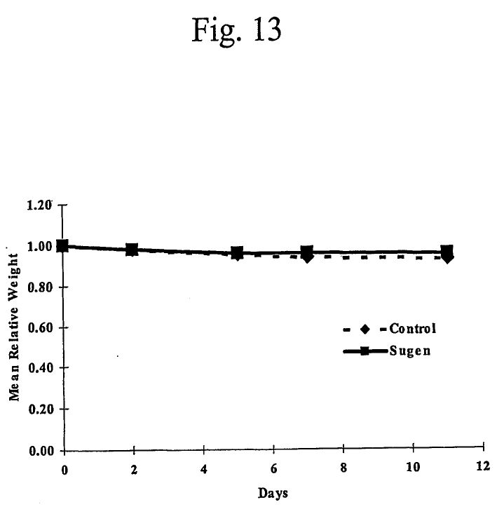

Chambers containing a matrix composition comprising fibrin were used to assess wound healing in response to systemic treatment with SUGEN 5416 at a concentration of 20 mg/kg. As shown in FIG. 13, no significant body loss in test animals as compared to control (i.e., untreated) animals was observed with the SUGEN 5416 treatment and chamber implantation. Indeed, the mean relative weight for the test animals closely followed the mean relative weight of the control animals over the 10-12 day test period.

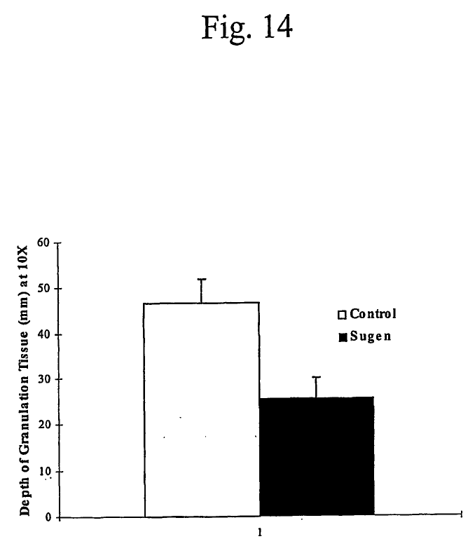

SUGEN 5416 inhibited neovascularization in the fibrin-containing chambers. This observation was made upon inspection of the gross appearance of the chambers from treated animals as compared to those from control animals, and upon inspection of hematoxylin and eosin (H & E) top sections and cross sections. It was also observed that SUGEN 5416 inhibited neovascularization in the fibrin-containing chambers, or F-ZC. Referring now to FIG. 14, the inhibition of neovascularization was confirmed by review of average MVD at 200× magnification. As shown in FIG. 14, an average MVD of about 30 was observed for the SUGEN test animals as compared to an average MVD of about 45 for the control animals, producing a p value of 0.0009.

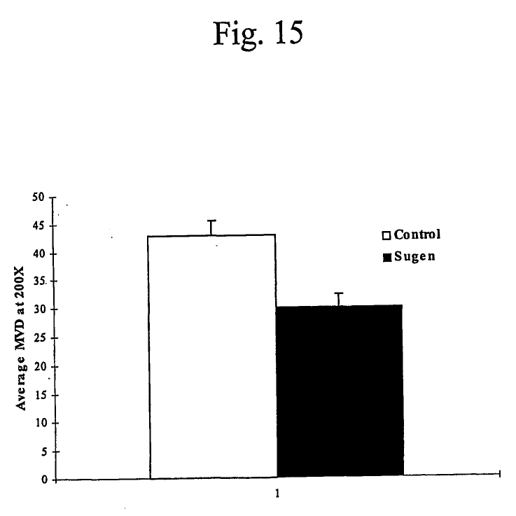

Referring now to FIG. 15, it was observed that in animals treated with SUGEN 5416, the formation of granulation tissue in the fibrin chambers F-ZC was inhibited, with a p value=0.0076. As shown by observation of the depth of granulation tissue at a magnification of 10×, an average depth of granulation of about 25 mm was observed for the SUGEN test animals, while an average depth of granulation tissue for the control animals was about 45 mm.

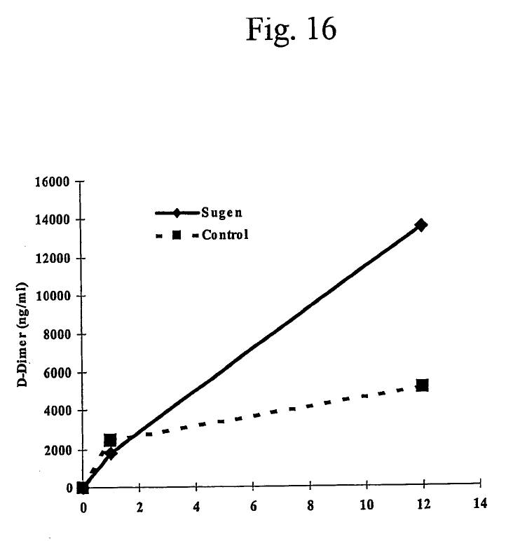

Referring now to FIG. 16, an increase in residual D-dimer retention was observed in fibrin-containing chambers F-ZC harvested from animals that were subjected to the SUGEN 5416 treatment as compared to control animals. Particularly, an increase in D-dimer levels to about 13,000 ng/ml was observed in the chambers from test animals as compared to D-dimer levels of about 4,000 ng/ml in the chambers from control animals after 10-12 days of implantation. The data presented in FIGS. 13-16 thus also establish an aspect of the utility of the chambers of the present invention in analyzing the effects of an angiogenesis-inhibiting agent in an in vivo setting.

### Laboratory Examples 7-9

Angiogenesis, development of new blood vessels, is essential for wound healing and tumor growth. A potentially important side effect of anti-angiogenic therapy can be delayed wound healing. In accordance with the present invention, these Examples investigate this side effect by using a novel in vivo method utilizing fibrin-containing dual porous PLEXIGLAS® chambers (fibrin Z-chambers, referred to herein as F-ZC, and in the case of tumor examination, referred to herein as tumor Z-chambers, or T-ZC) to investigate wound healing in rats administered with SU5416 (inhibitor of Flk-1 and Flt-1, at 20 mg/kg IP). SU5416-treated F-ZCs developed 45% less granulation tissue (p=0.0076) and showed a 10% reduction in microvessel density (p=0.0009) than controls treated with drug carrier alone. The granulation tissue showed distinctly decreased collagen deposition (p=0.0006) in SU5416 treated animals that was associated with 90% reduction in active TGF β1 level.

It was also observed that tissue transglutaminase (TG), a cross-linking enzyme involved in TGF β1 activation and matrix stabilization, was inhibited by SU5416. These results suggest that SU5416 delays wound healing by reducing matrix synthesis and stabilization through inhibition of TGF β1 activation. This study was made feasible via the, development of a method and chamber of the present invention to study anti-angiogenic compounds that provides highly reproducible and quantitative results.

### Materials and Methods for Laboratory Examples 7-9

Animal Protocols. The Duke Institutional Animal Care and Use Committee approved all animal protocols.

Schedule and Dose of Drug Administration. Female Fischer 344 rats of an average weight of >150 grams were selected for these studies. They were kept in rooms with the temperature controlled to 24° C. on a 12-hour light-dark cycle with access to rodent chow and bottled tap water ad libitum. SU5416 was administered at a dose of 20 mg/kg intraperitoneal once a day. The treatment solution was prepared at a concentration of 10 mg/ml, dissolved in Dimethyl Sulfoxide (DMSO) (# D128-1, Fisher Scientific Company of Hampton, N.H.) and CREMOPHOR® el reagent (#C5135, Sigma Chemical Company of St. Louis, Mo.) vehicle (1:1). This 1:1 DMSO and CREMOPHOR® vehicle was used as the control solution. Daily injections were started 2 days before initial surgery for implantation of fibrin chambers of the present invention. Rats with fibrin chambers tolerated surgery and daily injections very well and both treated and control animals maintained their pre-treatment weights for the duration of the study.

Fibrin Z-Chambers (F-ZC): In accordance with the present invention, F-ZC were employed in a fibrin gel based in vivo assay. In F-ZC, fibrinogen and thrombin are added to a dual porous chamber through a port and chambers are then implanted in the subcutaneous tissue of the rats and harvested at day 12 post-implantation to assess the wound healing response generated due to presence of fibrin. These chambers are constructed from customized PLEXIGLAS® rings with internal diameter of 10 mm and have an access port drilled on the side. The two open surfaces are covered by nylon mesh (pore size 180 microns, # NY8H04700, Millipore, Mass.) glued to the rings. Fibrinogen (# 341578, CalBiochem, La Jolla, Calif.) was prepared in DMEM (# 11995-065, Gibco BRL, Rockville, Md.) at a concentration of 4 mg/ml and was converted to fibrin by addition of thrombin (#605160, CalBiochem, La Jolla, Calif.) inside the chambers.

Fischer 344 rats were anesthetized, hair removed using the clippers and the surface was surgically prepared. Two small midline skin incisions were made in the dorsal region about 4 cm apart. Fascia was blunt dissected and small pockets were created on both sides along the midline incision. Thus, four F-ZCs were implanted per animal.

20 F-ZCs (5 animals) were implanted for each group (treatment or control). The F-ZCs were harvested on day 12-post surgery. The tissues were cut out from the chamber and were either preserved in 10% formalin for paraffin embedding (2 chambers) and snap frozen in liquid nitrogen for western blots, ELISA and D-Dimer measurements (2 chambers). The maximum depth of granulation tissue inside the F-ZC was measured from the H&E tissue sections to assess the degree of wound healing response. Two independent pathologists in a blinded fashion did all measurements.

Immunohistochemistry. Immunohistochemistry was carried out on paraffin embedded tissues for primary antibody against tissue transglutaminase (TG100, 1:10, endothelial cell marker (Haroon, Z. A., et al., Faseb J (1999) 13(13):1787-95), non-reactive to Factor XIIIa) (# MS-279, Neomarkers, Inc. of Union City, Calif.) or Isopeptide (#814 MAM, 1:75) CovalAB (Oullins, France) using procedures described previously. See Haroon, Z. A., et al., Faseb J (1999) 13(13):1787-95. Controls for the immunohistochemistry were treated with mouse IgG instead of primary antibody and were negative in any reactivity. Hematoxylin & Eosin (H&E) and Masson's trichrome (MT) were carried out as described by Sheehan (Sheehan, D., Hrapchak, B., Theory and practice of histotechnology. 2nd ed. 1980, Columbus, Ohio: Battelle Press) to evaluate collagen (green color) on the paraffin embedded tissue sections.

Quantitation of Immunohistochemistry. Microvessel density (MVD) was calculated as described by Weidner (Weidner, N., et al., J Natl Cancer Inst (1992) 84(24):1875-87). Briefly, three hot spots or areas with highest visible blood vessels density (marked by the vessel marker) per sample section were selected and number of blood vessels counted at a high power field (400×). The data was then pooled for the control and treated tissues to arrive at the mean values for each group. Collagen was semi-quantitatively estimated on a scale of 0-4. Zero was described as negligible staining, 1 as weak, 2 as moderate, 3 as strong and 4 as complete staining as observed in dermis. Two independent pathologists in a blinded fashion did all measurements.

Western Blot. The chamber contents were pooled for each group (treatment or control). Thus, each blot represents an average response from 10 different chambers (5 animals). Harvested chamber tissues were homogenized in cold lysis buffer (250 μl of lysis buffer was used per chamber, 10 chambers required 2.5 mls of lysis buffer) containing the proteolytic inhibitor cocktail (#1697498, Boehringer Mannheim, Mannheim, Germany) followed by sonification. They were then centrifuged and supernatant was removed and protein content was determined using a kit available from Bio-Rad of Hercules, Calif. Gel electrophoresis of the extracted tissue samples (100 μg) for TG antigen (TG100, 1:1000 dilution, # MS-279, Neomarkers, Inc. of Union City, Calif.) was carried out as described previously, see Haroon, Z. A., et al., Faseb J (1999) 13(13):1787-95. The amount of protein on the blot was estimated with a densitometer.

TGF β1 ELISA. Active TGF β1 ELISA was carried out in triplicate using DUOSET® kit (DY240, Genzyme Corporation, Cambridge, Mass.) as described previously (Danielpour, D., J Immunol Methods (1993) 158(1):17-25) with tissue lysates from chamber contents generated as detailed above. Latent form of TGF β31 was converted to immunoreactive form to ascertain total TGF β31 content. The data is shown as pg per mg of protein in the tissues.

D-Dimer Measurements. The MINIQUANT™ D-Dimer (Cat # 1447, Biopool AB of Umea, Sweden) assay was used to measure residual D-Dimer in the chamber content lysates generated as detailed above. The results are displayed in ng/ml. The measurements were carried out in duplicate. Each measurement is from pooled tissue for the treatment and control groups.

Microtiter Plate TG Assay. TG activity was determined by quantitating the incorporation of 5-biotin (amido) pentylamine into N, N′ -dimethyl casein coated microtiter plate as described previously. See Slaughter T. F., Anal Biochem (1992) 205(1):166-71.

Statistics. One-way ANOVA with Dunnett's post test was performed using GRAPHPAD INSTAT™ version 3.00 for Windows 95, GraphPad Software, San Diego Calif. USA, www.graphpad.com. All data is shown with±Standard error of the mean bars.

### Laboratory Example 7

### Delayed Wound Healing and Decreased MVD in F-ZC

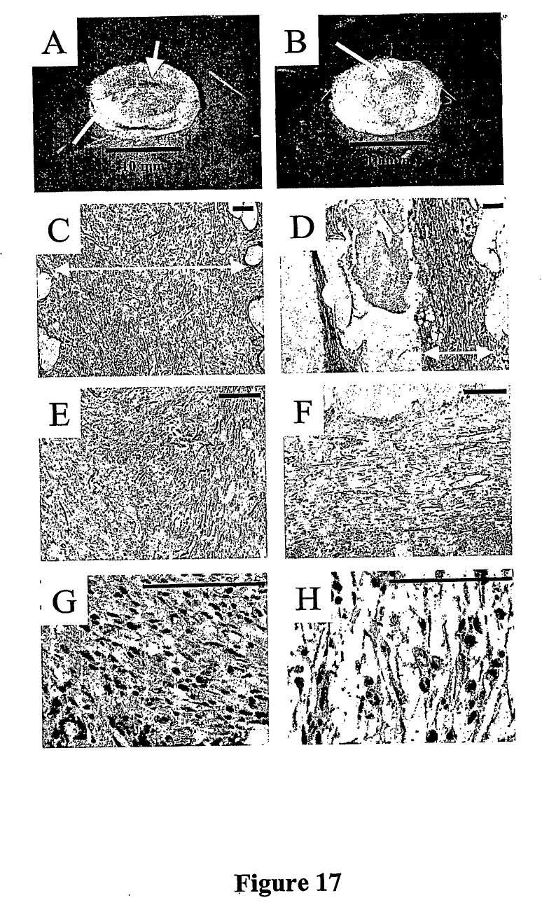

The fibrin inside the F-ZC initiates a wound healing response that results in production of granulation tissue. Dvorak, H. F., N Engl J Med (1986) 315(26):1650-9. On inspection upon harvest at day 12, SU5416 treated F-ZC showed distinctly paler color (FIGS. 17A and 17B) than controls (more red would indicate influx of blood vessels and red blood cells since fibrin itself is pale yellow). The depth of granulation tissue developed inside the SU5416 treated F-ZC was reduced by 45% compared with controls (FIGS. 17C and 17D, Table 1). The majority of the treated F-ZC developed granulation tissue non-homogenously inside the chamber. The MVD in the granulation tissue of SU5416 treated F-ZC dropped significantly by 10% from an average value of 30 microvessels per unit area of control tissues (Table 1). This observation is consistent with other reports of minor reduction in MVD with marked anti-tumor activity of anti-angiogenic compounds. Lund, E. L., Bastholm, L., and Kristjansen P. E., Clin Cancer Res (2000) 6(3):971-8.

### Laboratory Example 8

### Decreased Collagen Production is Due to Low level of Active TGF β1

The granulation tissue showed thinned out matrix in treated F-ZC. MT was utilized to assess collagen deposition and in semi-quantitative measurements observed significant decrease (>70%) with controls (FIGS. 17E and 17F, Table 1). Collagen production is primarily mediated by the pro-fibrogenic cytokine TGF β1. VEGF is known to induce TGF β1 (Saadeh, P. B., et al., Am J Physiol (1999) 277(4 Pt 1):C628-37) and it was expected SU5416 mediated inhibition of VEGF signal transduction would reduce TGF β1 production. The levels of total and active TGF β1 in the F-ZC tissue were measured by ELISA and it was found that active TGF β1 was reduced 90% even though the total TGF β1 was increased in treated F-ZC (Table 1).

### Laboratory Example 9

### TG is Potentially Responsible for Decreased Activation of TGF β1

TGF β1 is activated from its latent to active form predominantly by a surface complex of uPAR, plasminogen, mannose-6-phosphate receptor and TG. Kojima, S., Nara, K., and Rifkin, D. B., J Cell Biol (1993) 121(2):439-48. It was investigated which part of this pathway was being effected by SU5416. It was hypothesized that since plasmin production is up-regulated by VEGF (Baker, E. A., Bergin, F. G., and Leaper, D. J., Mol Pathol (2000) 53(6):307-12), VEGF RTKs inhibition would lead to reduced plasmin levels although there are leads that suggest VEGF induction of plasmin production is independent of RTKs. See Kroon, M. E., et al., Thromb Haemost (2001) 85(2):296-302. The fibrin inside the chambers is removed by plasmin producing D-Dimer (a fibrin degradation product), thus D-Dimer levels in the chambers would reflect the degree of plasmin activity. The D-Dimer values in SU5416 treated tissues remained elevated (Table 1). The relatively high levels of D-Dimer in the treated group suggested that plasmin production was not the target of inhibition by SU5416.

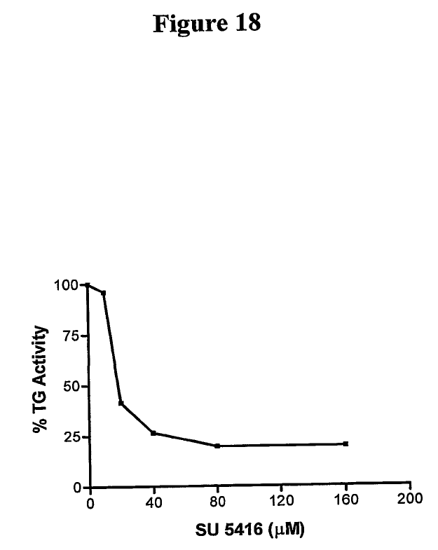

TG is a multi-functional wound healing enzyme with GTP and ATP binding sites. Greenberg, C. S., Birckbichler, P. J., and Rice, R. H., Faseb J (1991) 5(15):3071-7. TG cross-linking function (vital for TGF β1 activation) is calcium dependent and is inhibited by ATP/GTP binding. Lai, T. S., et al., J Biol Chem (1998) 273(3):1776-81; Lai, T. S., et al., J Biol Chem (1996) 271(49): 31191-5. It was postulated that SU5416 was reacting with ATP binding site, thereby inhibiting its cross-linking function. The interaction of SU5416 with TG was directly investigated. It was observed that SU5416 inhibited TG activity by more than 60% starting at 20 μM and reaching peak inhibition of 80% at 40 μM (FIG. 18).

The tissues were also probed with a monoclonal antibody directed towards the isopeptide bonds generated by TG to ascertain TG activity in the granulation tissue. Factor XIIIa can also generate isopeptide bonds in the granulation tissue (Gibran, N. S., Heimbach, D. M., and Holbrook, K. A., J Surg Res (1995) 59(3):378-86), but this would be limited to blood vessels and macrophages (sites of both Factor XIII and TG activity). Decreased TG activity was expected to lead to low levels of isopeptide bonds in the ECM. Very low levels of isopeptide bonds were detected in the extracellular matrix of SU6416 treated tissues in comparison to controls, confirming this hypotheses (FIGS. 17G and 17H).

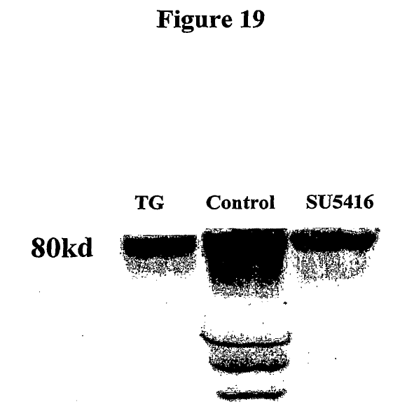

The molecular form of TG antigen in the chamber tissues was next investigated. It was observed that TG production was decreased by more than 70% and the TG antigen was not proteolyzed in SU5416 treated tissues in western blot results (FIG. 19, Table 1). It has been reported earlier that TG is degraded in healing tissues (Haroon, Z. A., et al., Faseb J (1999) 13(13):1787-95) and TG degradation by trypsin is inhibited when the ATP/GTP binding sites are occupied (Lai, T. S., et al., J Biol Chem (1998) 273(3):1776-81). The relative lack of TG degradation suggests that SU5416 could have interacted with the ATP/GTP binding site of TG, shutting down the cross-linking function.

One of the critical problems in studying effects of anti-angiogenic compounds on healing has been the absence of reliable animal models to carry out such studies. In accordance with the present invention, a novel fibrin chamber and assay has been developed to provide such a model. This model is rapid, involves minimal animal usage, is reproducible, allows both chamber & systemic administration of compounds and both wound healing & tumor growth can be studied.

Table 1 presents a summary of various measurements carried out on paraffin embedded F-ZC and tissue lysates obtained from F-ZC. Data is presented with±standard error of mean values. Please refer to Examples 7-9 for experimental details and data generation.

TABLE 1

| Measurement | Control | SU5416 | Difference |
|---|---|---|---|
| Granulation | 466.7 ± 51.9 | 256.8 ± 42.9 | 45%, p = 0.006, |
| Microvessel | 28.17 ± 0.42 | 26.25 ± 0.53 | 10%, p = 0.006, |
| Collagen | 1.75 ± 0.25 | 0.5 ± 0.18 | 72%, p = 0.001, |
| D-Dimer (ng/ml) | 4100 ± 100 | 185 ± 20 | 96%, n = 10 |
| Active TGF β1 | 1.45 ± 0.1 | 0.11 ± 0.05 | 93%, n = 10 |
| Latent TGF β1 | 7.72 ± 0.5 | 11.85 ± 0.8 | 35%, n = 10 |
| TG (Western | 100% | 29% | 71%, n = 10 |
| TG Fragments | 56% | 0% | n = 10 (pooled) |

### Laboratory Example 10

### Assessment of Wound Healing Response with Chamber Administration of Angiostatin (1 uM)

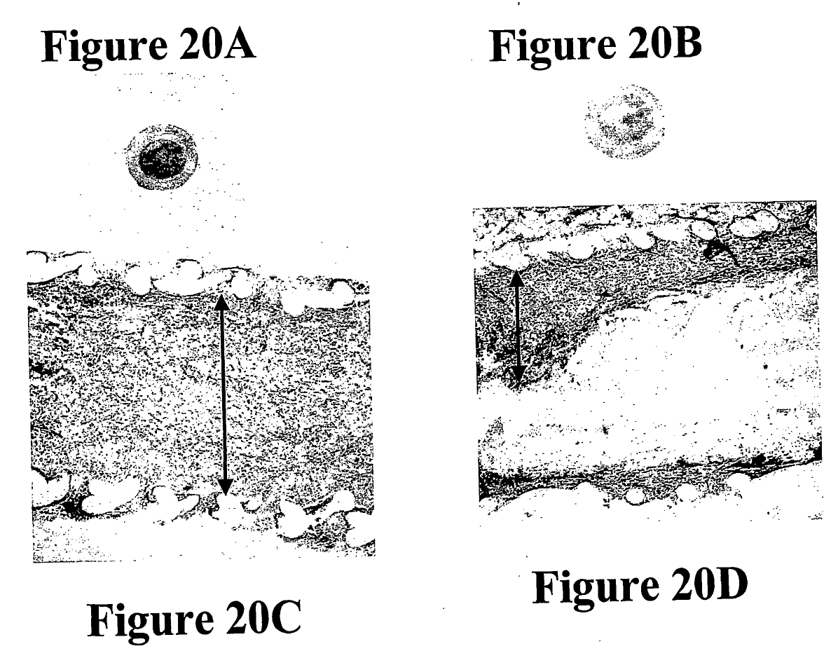

FIGS. 20A and 20B are photographs showing gross examination of fibrin-containing chambers of the present invention in which angiostatin was administered in laboratory animals in accordance with techniques described herein above for Laboratory Examples 1-9. FIGS. 20A and 20B show more influx of blood vessels in the controls (FIG. 20A) than angiostatin treated chambers (1 μM angiostatin), which appeared paler in color (FIG. 20B). Fibrin is inherently pale yellow in color and lack of blood vessels in angiostatin treated chambers results in the paler appearance of the chambers.

FIGS. 20C and 20D are photomicrographs showing depth (represented by line with arrowheads at each end) of granulation tissue developed inside fibrin-containing chambers of the present invention. Depth granulation was used as a measure for the healing response. The granulation tissue in controls (FIG. 20C) is distinctly more than angiostatin treated chambers (1 μM angiostatin) (FIG. 20D). A reduction in healing response in the presence of angiostatin was thus observed.

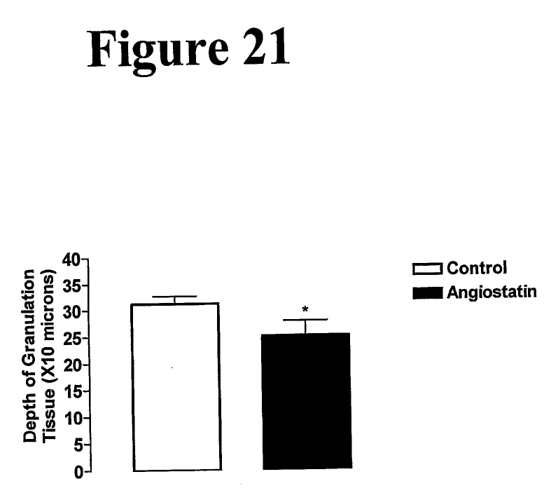

FIG. 21 is a bar graph depicting that angiostatin inhibited granulation tissue formation in fibrin-containing chambers of the present invention implanted in test animals (shaded bar) as compared to fibrin-containing chambers of the present invention implanted in control animals (open bar). Scale=depth of granulation tissue (×10 microns).

### Laboratory Example 11

### Assessment of Effect of SOD Mimetics on Tumor Growth

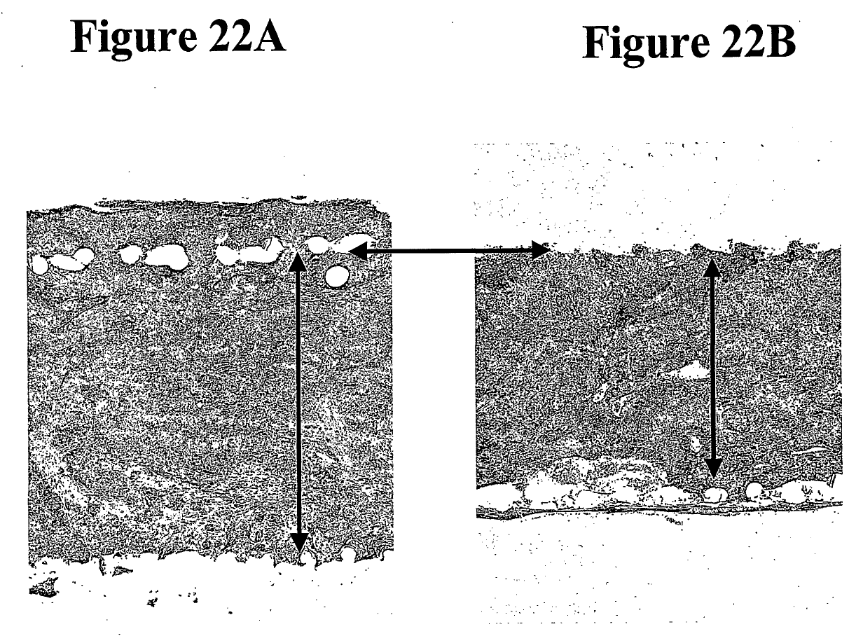

FIGS. 22A and 22B are photomicrographs showing depth (represented by line with arrowheads at each end) of granulation tissue developed inside fibrin-containing chambers of the present invention in which superoxide dismutase (SOD) mimetics were administered to tumors in laboratory animals in accordance with techniques described herein above for Laboratory Examples 1-9. In this Example the chambers of the present invention are referred to as tumor Z-Chambers, or T-ZC. This Example was performed to assess the predictive value of fibrin-containing T-ZCs of the present invention where tumor reduction was expected to be minor (e.g. about a 15% reduction).

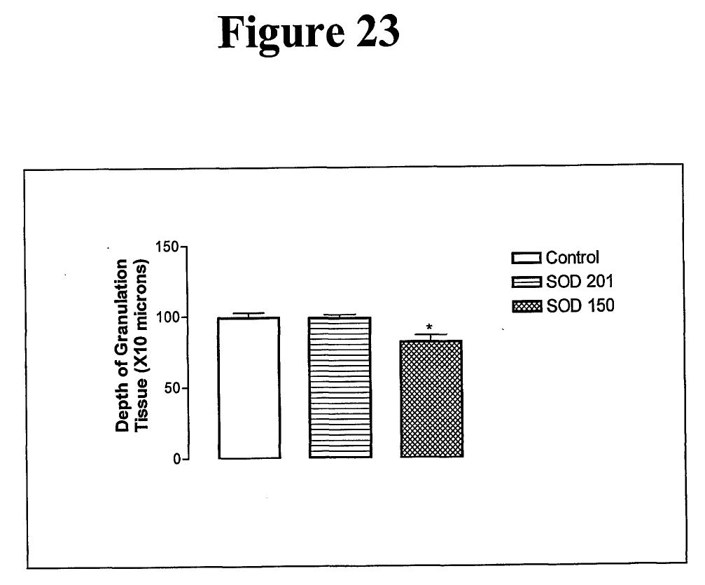

FIG. 23 is a bar graph depicting the detection of a minor reduction in tumor growth after SOD mimetic administration. This reduction was detected T-ZCs of the present invention implanted in test animals (horizontally hatched bar=SOD 201; cross hatched bar-SOD 150) as compared to T-ZCs of the present invention implanted in control animals (open bar). Scale=depth of granulation tissue (×10 microns); p<0.01.

### REFERENCES

The publications and other materials listed below and/or set forth by author and date in the text above to illuminate the background of the invention, and in particular cases, to provide additional details respecting the practice, are incorporated herein by reference. Materials used herein include but are not limited to the following listed references.

Ausprunk et al., Am. J. Pathol., 79:597-618 (1975).

Baker, E. A., Bergin, F. G., and Leaper, D. J., Mol Pathol (2000) 53(6):307-12.

Dvorak, H. F., et al., Laboratory Investigation 57(6): pp. 673-686 (1987).

Dvorak, H. F., N Engl J Med (1986) 315(26):1650-9.

Folkman, J., N Engl J Med 28;333(26), 1757-1763 (1995).

Gibran, N. S., Heimbach, D. M., and Holbrook, K. A., J Surg Res (1995) 59(3):378-86.

Greenberg, C. S., Birckbichler, P. J., and Rice, R. H., Faseb J (1991) 5(15):3071-7.

Haroon, Z. A., et al., Faseb J (1999) 13(13):1787-95.

Kojima, S., Nara, K., and Rifkin, D. B., J Cell Biol (1993) 121(2):439-48.

Kroon, M. E., et al., Thromb Haemost (2001) 85(2):296-302.

Lai, T. S., et al., J Biol Chem (1996) 271(49): 31191-5.

Lai, T. S., et al., J Biol Chem (1998) 273(3):1776-81.

Lund, E. L., Bastholm, L., and Kristjansen P. E., Clin Cancer Res (2000) 6(3):971-8.

Mendel et al., (2000) Anti-Cancer Drug Design 15:29-41.

Ossonski et al., (1980) Cancer Res., 40:2300-2309.

Remington's Pharmaceutical Sciences, 16th Ed. Mack Publishing Company (1980).

Saadeh, P. B., et al., Am J Physiol (1999) 277(4 Pt 1):C628-37.

Sambrook et al. (1992) Molecular Cloning: A Laboratory Manual (Cold Spring Harbor Laboratory Press, Cold Spring Harbor, N. Y. ).

Sheehan, D., Hrapchak, B., Theory and practice of histotechnology. 2nd ed. 1980, Columbus, Ohio: Battelle Press.

Slaughter T. F., Anal Biochem (1992) 205(1):166-71.

U.S. Pat. No. 5,792,783.

Weidner, N., et al., J Natl Cancer Inst (1992) 84(24):1875-87.

It will be understood that various details of the invention may be changed without departing from the scope of the invention. Furthermore, the foregoing description is for the purpose of illustration only, and not for the purpose of limitation—the invention being defined by the claims.

What is claimed is:

1. A chamber for in vivo delivery of an active agent, the chamber comprising:
(a) a housing having at least two porous surfaces, the at least two porous surfaces disposed on substantially opposite sides of the housing from each other;
(b) an internal void space within the housing; and
(c) a matrix composition comprising an active agent, the matrix composition disposed within the internal void space.

2. The chamber of claim 1, wherein the housing further comprises a support disposed between the at least two porous surfaces.

3. The chamber of claim 2, wherein the support comprises a ring, the ring contacting the at least two porous surfaces along a periphery of the at least two porous surfaces.

4. The chamber of claim 3, wherein the ring has an inner diameter ranging from about 5 to about 15 millimeters.

5. The chamber of claim 2, further comprising a port on a surface of the support, the port opening to the internal void space.

6. The chamber of claim 5, wherein the port has a diameter ranging from about 1 micrometer to about 3 micrometers.

7. The chamber of claim 1, wherein the at least two porous surfaces further comprise a mesh.

8. The chamber of claim 7, wherein the mesh further comprises a pore size ranging from about 150 to 200 micrometers.

9. The chamber of claim 1, wherein the active agent comprises a cell, a tissue growth modulating agent or combinations thereof.

10. The chamber of claim 9, wherein the cell comprises a cell from a target tissue.

11. The chamber of claim 9, wherein the tissue growth modulating agent is fibrin, fibrinogen, transforming growth factor, or combinations thereof.

12. The chamber of claim 1, wherein the matrix composition further comprises a stabilizing agent.

13. A fibrin matrix composition, comprising:
(a) an effective amount of fibrin; and
(b) a stabilizing agent in an amount sufficient to retard degradation of fibrin in an in vivo setting.

14. The fibrin matrix composition of claim 13, wherein the stabilizing agent is a protease inhibitor.

15. The fibrin matrix composition of claim 14, wherein the protease inhibitor is selected from the group consisting of PMSF, N-caproic acid, plasminogen inhibiting factor 1, plasminogen inhibiting factor 2, aprotnin, and combinations thereof.

16. The fibrin matrix composition of claim 15, wherein the matrix composition further comprises PMSF at a concentration ranging from about 0.5 μM to about 50 μM, and N-caproic acid at a concentration ranging from about 1 mM to about 100 mM.

17. A method of screening a candidate compound for tissue growth modulating activity, the method comprising:
(a) providing a chamber comprising:
(i) a housing having at least two porous surfaces, the at least two porous surfaces disposed on substantially opposite sides of the housing from each other;
(ii) an internal void space within the housing; and
(iii) a matrix composition comprising a tissue growth modulating agent, the matrix composition disposed within the internal void space;
(b) implanting the chamber into an animal subject;
(c) administering a candidate compound to the animal subject;
(d) extracting the chamber after a time suitable for measurement of tissue growth; and
(e) evaluating tissue growth in the chamber to thereby determine the tissue growth modulating activity of the candidate compound.

18. The method of claim 17, wherein the tissue growth modulating activity comprises angiogenesis modulating activity.

19. The method of claim 17, wherein the housing of the chamber further comprises a support disposed between the at least two porous surfaces.

20. The method of claim 19, wherein the support comprises a ring, the ring contacting the at least two porous surfaces along a periphery of the at least two porous surfaces.

21. The method of claim 20, wherein the ring has an inner diameter ranging from about 5 to about 15 millimeters.

22. The method of claim 19, further comprising a port on a surface of the support, the port opening to the internal void space.

23. The method of claim 22, wherein the port has a diameter ranging from about 1 micrometer to about 3 micrometers.

24. The method of claim 17, wherein the at least two porous surfaces comprise a mesh.

25. The method of claim 24, wherein the mesh further comprises a pore size ranging from about 150 to 200 micrometers.

26. The method of claim 17, wherein the tissue growth modulating agent is fibrin, fibrinogen, transforming growth factor, or combinations thereof.

27. The method of claim 17, wherein the matrix composition further comprises a cell.

28. The method of claim 27, wherein the cell comprises a cell from a neoplasm.

29. The method of claim 17, wherein the test animal further comprises a neoplasm, and the chamber is implanted in the neoplasm.

30. The method of claim 17, further comprising implanting two or more chambers into the test animal.

31. The method of claim 17, wherein the candidate compound is systemically administered to the animal subject.

32. The method of claim 17, wherein the candidate compound is administered in the chamber.

33. The method of claim 17, further comprising implanting two or more test chambers into the test animal, wherein a different candidate compound is inserted in each chamber.

34. The method of claim 17, wherein the candidate compound is administered to the test animal by:
(i) collecting serum from a human subject at a time after the human subject received a candidate tissue growth modulating compound;
(ii) adding the serum to the chamber; and
(iii) implanting the chamber in the animal subject.

35. The method of claim 17, wherein the chamber is incubated in the test animal for about 5 to about 15 days.

36. The method of claim 17, wherein the evaluating of tissue growth is accomplished by a technique selected from the group consisting of histology, immunohistochemistry, confocal imaging, magnetic resonance imaging, assessment of tumor growth, assessment of vascular density, immunoblotting, assessment of cell migration rate, assessment of cell death, assessment of hypoxia, assessment of vascular permeability and combinations thereof.

37. A method of generating tissue growth in a vertebrate animal, the method comprising:
(a) providing a chamber comprising:
(i) a housing having at least two porous surfaces, the at least two porous surfaces disposed on substantially opposite sides of the housing from each other;
(ii) an internal void space within the housing; and
(iii) a matrix composition comprising an tissue growth modulating agent, the matrix composition disposed within the internal void space; and
(b) implanting the chamber in the vertebrate animal; and
(c) generating tissue growth in the vertebrate animal through the implanting of the chamber.

38. The method of claim 37, wherein the housing of the chamber further comprises a support disposed between the at least two porous surfaces.

39. The method of claim 38, wherein the support comprises a ring, the ring contacting the at least two porous surfaces along a periphery of the at least two porous surfaces.

40. The method of claim 39, wherein the ring has an inner diameter ranging from about 5 to about 15 millimeters.

41. The method of claim 39, further comprising a port on a surface of the support, the port opening to the internal void space.

42. The method of claim 41, wherein the port has a diameter ranging from about 1 micrometer to about 3 micrometers.

43. The method of claim 37, wherein the at least two porous surfaces comprise a mesh.

44. The method of claim 43, wherein the mesh further comprises a pore size ranging from about 150 to 200 micrometers.

45. The method of claim 37, wherein the tissue growth modulating agent is fibrin, fibrinogen, transforming growth factor, or combinations thereof.

46. The method of claim 45, wherein the matrix composition further comprises a stabilizing agent in an amount sufficient to retard degradation of the tissue growth modulating agent.

47. The method of claim 37, wherein the matrix composition further comprises a cell.

48. The method of claim 37, wherein the vertebrate animal is a warm-blooded vertebrate animal.

49. The method of claim 48, wherein the warm-blooded vertebrate animal is a mammal.

50. The method of claim 37, where the implanting of the chamber further comprises implanting the chamber in the vertebrate animal in a target tissue.

51. The method of claim 37, wherein the tissue growth that is modulated comprises angiogenesis.
